(19) United States
(12) Patent Application Publication
(10) Pub. No.: US 2016/0207903 A1
(43) Pub. Date: Jul. 21, 2016

(54) Photochromic Compounds

(71) Applicant: Chromatic Technologies, Inc., Colorado Springs, CO (US)

(72) Inventors: Qi Wang, Colorado Springs, CO (US); Ruizheng Wang, Colorado Springs, CO (US); Medhanei Abraha, Colorado Springs, CO (US)

(73) Assignee: Chromatic Technologies, Inc., Colorado Springs, CO (US)

(21) Appl. No.: 14/997,806

(22) Filed: Jan. 18, 2016

Related U.S. Application Data
(60) Provisional application No. 62/105,157, filed on Jan. 19, 2015.

(51) Int. Cl. C07D 311/92; C09D 11/50; C07D 405/04; C09K 9/02

(52) U.S. Cl. CPC C07D 311/92; C09K 9/02; C09D 11/50; C07D 405/04; C09K 2211/1088; C09K 2211/1011; C09K 2211/1007; C09K 2211/1029; C09K 2211/1044

(57) ABSTRACT

Described are novel photochromic naphthopyran compounds which exhibit a red coloring when in a red-colored state achieved upon exposure to ultraviolet light, whereby the red coloring has a lambda max in a range of between about 490 nanometers to about 510 nanometers or between about 490 nanometers to about 500 nanometers.

This United States Non-Provisional patent application claims the benefit of U.S. Provisional Patent Application No. 62/105,157, filed Jan. 19, 2015, hereby incorporated by reference herein.

### I. BACKGROUND

Photochromic compounds exhibit a reversible change in color when exposed to light radiation involving ultraviolet rays, such as the ultraviolet radiation in sunlight or the light of a mercury lamp. When the ultraviolet radiation is discontinued, or as to particular photochromic compounds, upon an increase in temperature, the photochromic compound will return to its original color or a colorless state.

For applications in which a sunlight-induced reversible color change is desired, there are various conventional photochromic compounds which exhibit a variety of colors when in a colored state achieved upon exposure to ultraviolet light. However, none of these conventional photochromic compounds exhibit a red coloring having a lambda max in a range of between about 490 nanometers to about 510 nanometers or between about 490 nanometers to about 500 nanometers, in the colored state.

### II. SUMMARY OF THE INVENTION

Accordingly, a broad object of a particular embodiment of the invention can be to provide a novel photochromic naphthopyran compound which exhibits a red coloring when in a red-colored state achieved upon exposure to ultraviolet light, whereby the red coloring has a lambda max in a range of between about 490 nanometers to about 510 nanometers or between about 490 nanometers to about 500 nanometers.

Naturally, further objects of the invention are disclosed throughout other areas of the specification, drawing, and claims.

### III. A BRIEF DESCRIPTION OF THE FIGURES

The application file contains at least one FIGURE executed in color. Copies of this patent application publication with color figure(s) will be provided by the Office upon request and payment of the necessary fee.

FIG. 1 illustrates a red coloring exhibited by the particular novel naphthopyran compound having formula II, dissolved in a polymer matrix, when in a red-colored state achieved upon exposure to ultraviolet light. For comparison, FIG. 1 additionally illustrates a red-like coloring exhibited by PHOTOSOL® 7-49, a conventional red-like photochromic compound having formula III, dissolved in a polymer matrix, when in a red-like-colored state achieved upon exposure to ultraviolet light.

### IV. DETAILED DESCRIPTION OF THE PREFERRED EMBODIMENTS

The present invention relates to novel naphthopyran compounds having photochromic properties, whereby the novel naphthopyran compounds exhibit a red coloring when in a red-colored state achieved upon exposure to ultraviolet radiation. Following discontinuation of the ultraviolet radiation or, as to particular embodiments, upon an increase in temperature, the novel naphthopyran compounds reversibly transition from the red-colored state to a non-red-colored state, in which the novel naphthopyran compounds exhibit a non-red coloring or are colorless.

The term “red” for the purposes of the present invention means a coloring having a lambda max or a wavelength of greatest absorbance in a range of between about 490 nanometers to about 510 nanometers or between about 490 nanometers to about 500 nanometers.

The term “non-red” for the purposes of the present invention means a coloring having a lambda max or a wavelength of greatest absorbance outside of the range of between about 490 nanometers to about 510 nanometers, between about 490 nanometers to about 500 nanometers, or a colorless coloring.

The novel naphthopyran compounds of the present invention exhibit a reversible color change having greater color density when in a red-colored state in relation to the lesser color density exhibited by conventional red-like photochromic compounds, for example PHOTOSOL® 7-49, when in a red-like-colored state. Additionally, the novel naphthopyran compounds of the present invention exhibit rapid coloration and discoloration kinetics, sufficient thermal stability, and sufficient photostability.

Accordingly, the novel naphthopyran compounds of the present invention may be useful as either a single compound to provide a red-colored state or in combination with other compounds, whether photochromic compounds or non-photochromic colorants, to provide additional colored states. As an illustrative example, the novel naphthopyran compounds of the present invention can be combined with compounds having lambda maxes which provide other primary colors in their colored states, to achieve a desired colored state. As another illustrative example, the novel naphthopyran compounds of the present invention can be combined with compounds having lambda maxes which provide other colors (such as secondary, tertiary, quarternary, or other colors) in their colored states, to achieve a desired colored state.

As used herein, the term “combination or combining” refers to any method of putting two or more materials together. Such methods include, but are not limited to, mixing, blending, commingling, concocting, homogenizing, ultrasonic homogenizing, incorporating, intermingling, fusing, joining, shuffling, stirring, coalescing, integrating, confounding, uniting, creating a solution of two or more materials, creating a suspension of two immiscible materials, such as an emulsion, via any number of means, or the like, or combinations thereof.

The novel naphthopyran compounds of the present invention have the following formula I:

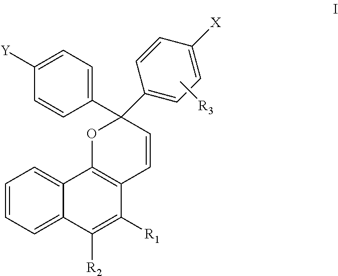

wherein:

R₁, R₂, and R₃ are each independently selected from acyloxy, alkoxy, alkyl, amine, aryl, carbonyl, halo, heterocyclic, hydrogen, nitrile, and nitro;

X is independently selected from alkenyl, alkoxy, alkyl, alkynyl, amine, aryl, halo, and hydrogen; and

Y is independently selected from alkoxy, alkyl, amine, and hydrogen.

As to particular embodiments, whether in R₁, R₂, R₃, X, or Y, the acyloxy, alkenyl, alkoxy, alkyl, alkynyl, amine, aryl, or carbonyl groups can be either unsubstituted or substituted.

For the purposes of the present invention, the term “substituted” means having a substituent comprising any element other than hydrogen or any group of elements. As to particular embodiments, the substituent can be further substituted one or more times. Particular substituents may be selected to attain the desired properties for a specific application and can include, for example, hydrophobic groups, hydrophilic groups, solubilizing groups, blocking groups, or leaving groups.

As to particular embodiments of one or more novel naphthopyran compounds, R₁ can affect coloration kinetics, discoloration kinetics, or both.

As to particular embodiments of one or more novel naphthopyran compounds, R₂ can affect coloration kinetics, discoloration kinetics, or both.

As to particular embodiments of one or more novel naphthopyran compounds, X can affect lambda max, color density, or both. Additionally, X can affect coloration kinetics, discoloration kinetics, or both.

As to particular embodiments of one or more novel naphthopyran compounds, Y can affect lambda max, color density, or both. Additionally, Y can affect coloration kinetics, discoloration kinetics, or both.

As an illustrative example, a novel naphthopyran compound of the present invention can be ethyl 6-acetoxy-2-(4-bromophenyl)-2-(4-propoxyphenyl)-2H-benzo[h]chromene-5-carboxylate, having the following formula II:

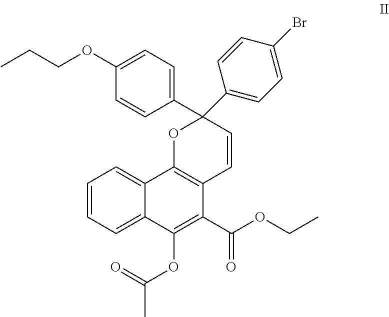

wherein:

R₁ is a carbonyl, specifically an ester having the ester carbon, which is singly bonded to a first oxygen and doubly bonded to a second oxygen, singly bonded directly to the corresponding ring;

R₂ is a carbonyl, specifically an ester having a first oxygen, which is singly bonded to the ester carbon, singly bonded directly to the corresponding ring;

R₃ is hydrogen;

X is bromine (Br); and

Y is alkoxy.

The novel naphthopyran compounds of the present invention can be prepared in a similar fashion to the synthetic process described below, which details the synthesis of the particular novel naphthopyran compound having formula II, as follows:

### Step 1:

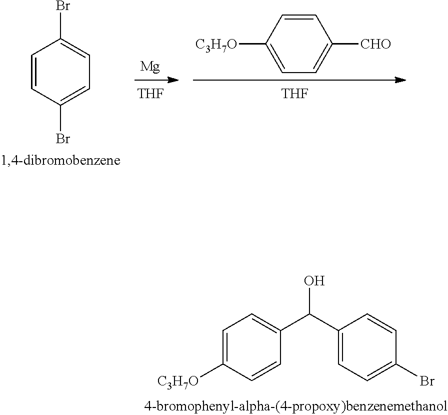

Step 1: Synthesis of 4-bromophenyl-alpha-(4-propoxy)benzenemethanol. 1,4-dibromobenzene (28.27 g, 120 mmol) in dry tetrahydrofuran (THF) (100 mL) can be combined with dry THF (80 mL) having magnesium (Mg) turnings (3.47 g, 144 mmol) at room temperature under nitrogen atmosphere to provide a first reaction mixture, which can then be stirred and refluxed for 1.5 h followed by cooling to room temperature. The first reaction mixture can then be combined with 4-propoxybenzaldehyde (C₃H₇OC₆H₄CHO) (16.4 g, 100 mmol) in dry THF (100 mL) at 0° C. to provide a second reaction mixture, which can then be warmed to room temperature and refluxed for 1.5 h. Next, saturated ammonium chloride (NH₄Cl) (250 mL) can be added to the second reaction mixture to quench the second reaction. Following separation, the organic layer can be collected and the aqueous layer can be extracted with ethyl acetate (3×50 mL) to provide additional organic layers. Subsequently, the organic layers can be combined and washed with brine (200 mL). Following, the combined organic layers can be dried over sodium sulfate (Na₂SO₄) and concentrated to provide a light yellow oil of 4-bromophenyl-alpha-(4-propoxy)benzenemethanol, which can have a yield of about 28.3 g (88%).

### Step 2:

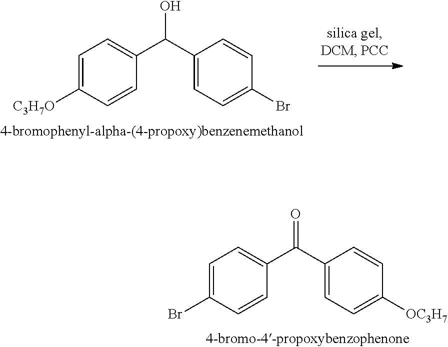

Step 2: Synthesis of 4-bromo-4′-propoxybenzophenone. To the 4-bromophenyl-alpha-(4-propoxy)benzenemethanol (28.3 g, 88.2 mmol) obtained from Step 1, silica gel (30 g) in dichloromethane (DCM) (450 mL) and pyridinium chlorochromate (PCC) (22.25 g, 103.2 mmol) can be added at 0° C. to provide a fourth reaction mixture, which can then be warmed to room temperature and stirred for 2 h. Next, the fourth reaction mixture can be filtered, whereby the filtrate can be washed with brine (200 mL) and concentrated to provide a filtrate residue, which can then be passed through a silica gel column and eluted with hexanes/DCM, 1/1, v/v. Following, the eluted filtrate residue can be recrystallized from the hexanes/DCM to provide white solids of 4-bromo-4′-propoxybenzophenone, which can have a yield of about 18 g (64%).

### Step 3:

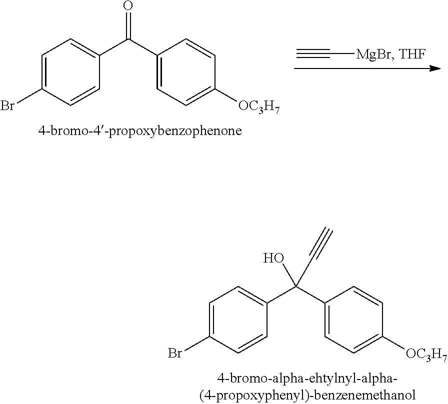

Step 3: Synthesis of 4-bromo-alpha-ethynyl-alpha-(4-propoxyphenyl)-benzenemethanol. To the 4-bromo-4′-propoxybenzophenone (8 g, 25 mmol) obtained from Step 2, dry THF (110 mL) can be added to provide a fifth reaction mixture. Subsequently, ethynylmagnesium bromide solution (CHCMgBr) (0.5 M in THF, 105 mL) can be added at 0° C. to provide a sixth reaction mixture, which can then be warmed to room temperature and stirred for 3 h. The sixth reaction can be monitored with thin layer chromatography (TLC), whereby after the 4-bromo-4′-propoxybenzophenone has been fully consumed, the sixth reaction can be quenched by the addition of saturated NH₄Cl (150 mL). Following separation, the organic layer can be collected and the aqueous layer can be extracted with ethyl acetate (3×40 mL) to provide additional organic layers. Next, the organic layers can be combined, dried over Na₂SO₄, and concentrated, whereby the residue can be purified by flash chromatography, eluting with hexanes/DCM, ˜2/1-1/1, v/v, to provide a yellow oil of 4-bromo-alpha-ethynyl-alpha-(4-propoxyphenyl)-benzenemethanol, which can have a yield of about 8.5 g (98%).

### Step 4:

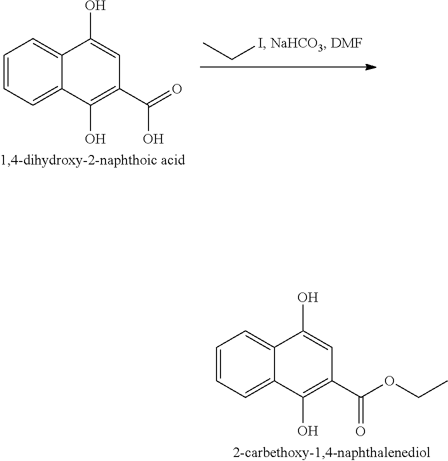

Step 4: Synthesis of 2-carbethoxy-1,4-naphthalenediol, which can be performed in any order in relation to Step 1 through Step 3, but before Step 5. 1,4-dihydroxy-2-naphthoic acid (50 g, 0.245 mol), sodium bicarbonate (NaHCO₃) (41 g, 0.49 mol), and dimethylformamide (DMF) (500 mL) can be combined to provide a seventh reaction mixture. Iodoethane (C₂H₆I) (19.6 mL, 0.245 mol) can be combined with the seventh reaction mixture at room temperature to provide an eighth reaction mixture, which can then be heated at 50° C. overnight. Next, the eighth reaction mixture can be quenched by the addition of 1M hydrogen chloride in an ice bath until the pH of the eighth reaction mixture alkalinizes to a pH of less than 2. Following, the eighth reaction mixture can be extracted with ethyl acetate (5×150 mL), whereby the organic layers can be combined, washed successively with brine (200 mL) and water (3×200 mL), and dried over Na₂SO₄. Subsequently, the combined organic layers can be concentrated and purified by flash chromatography, eluting with hexanes/ethyl acetate, ˜4/1-3/1, v/v. Following, the hexanes/ethyl acetate can be removed, for example via evaporation, and the product can be recrystallized from a hexanes/DCM solvent system to provide yellow solids of 2-carbethoxy-1,4-naphthalenediol, which can have a yield of about 35.5 g (63%).

### Step 5:

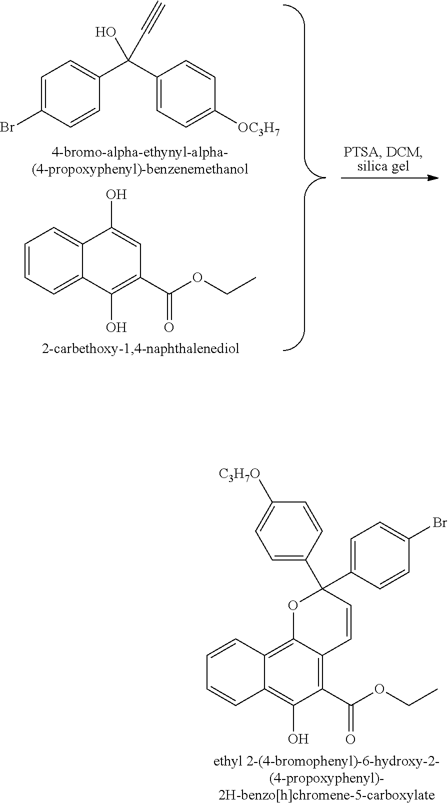

Step 5: Synthesis of ethyl 2-(4-bromophenyl)-6-hydroxy-2-(4-propoxyphenyl)-2H-benzo[h]chromene-5-carboxylate. 4-bromo-alpha-ethynyl-alpha-(4-propoxyphenyl)-benzenemethanol (4.65 g, 13.5 mmol) obtained from Step 3 and 2-carbethoxy-1,4-naphthalenediol (3.45 g, 14.8 mmol) obtained from Step 4 can be combined with p-toluenesulfonic acid (PTSA) (256 mg, 1.35 mmol), silica gel (15 g), and DCM (70 mL) to provide a ninth reaction mixture, which can then be rotated on a rotary evaporator at 45° C., whereby the solvent can be removed under slightly reduced pressure in about 30 min. The resultant solids can be purified by flash chromatography, eluting with hexanes/DCM, ˜3/1-2/1, v/v) to provide ethyl 2-(4-bromophenyl)-6-hydroxy-2-(4-propoxyphenyl)-2H-benzo[h]chromene-5-carboxylate, which can have a yield of about 5.37 g (71%).

### Step 6:

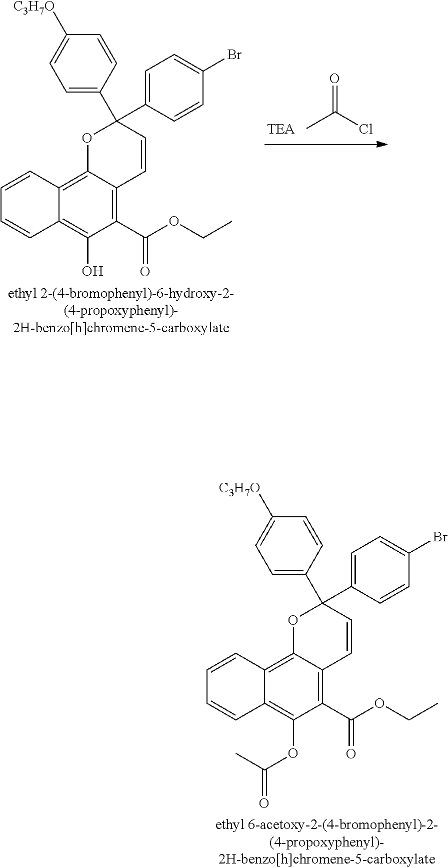

Step 6: Synthesis of ethyl 6-acetoxy-2-(4-bromophenyl)-2-(4-propoxyphenyl)-2H-benzo[h]chromene-5-carboxylate. To the ethyl 2-(4-bromophenyl)-6-hydroxy-2-(4-propoxyphenyl)-2H-benzo[h]chromene-5-carboxylate (3.00 g, 5.36 mmol) obtained from Step 5 and triethylamine (TEA) (1.1 mL, 11.8 mmol) in dry DCM (20 mL), acetyl chloride (CH₃COCl) (0.42 mL, 5.90 mmol) can be added at 0° C. to provide a tenth reaction mixture, which can then be warmed to room temperature and stirred for 2 h. The tenth reaction can be monitored with TLC, whereby after the ethyl 2-(4-bromophenyl)-6-hydroxy-2-(4-propoxyphenyl)-2H-benzo[h]chromene-5-carboxylate has been fully consumed, the tenth reaction mixture can be washed with saturated NH₄Cl (30 mL) and brine (30 mL). Next, the organic layer can be dried and concentrated, whereby the residue can be purified by flash chromatography, eluting with hexanes/DCM, 1/1, v/v) to provide pink solids of the novel naphthopyran compound, ethyl 6-acetoxy-2-(4-bromophenyl)-2-(4-propoxyphenyl)-2H-benzo[h]chromene-5-carboxylate, having formula II, which can have a yield of about 2.97 g (92%).

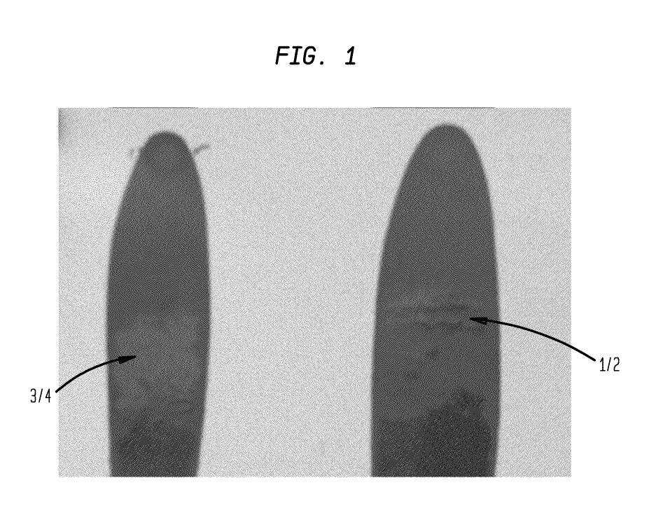

Now referring primarily to FIG. 1, which is a photograph illustrating a red coloring exhibited by the particular novel naphthopyran compound having formula II (1), dissolved in a polymer matrix, when in a red-colored state (2) achieved upon exposure to ultraviolet light. For comparison, FIG. 1 additionally illustrates a red-like coloring exhibited by PHOTOSOL® 7-49, a conventional red-like photochromic compound having formula III (3), dissolved in a polymer matrix, when in a red-like-colored state (4) achieved upon exposure to ultraviolet light. PHOTOSOL® 7-49 can be obtained from PPG Industries, Inc., 440 College Park Drive, Monroeville, Pa. 15146.

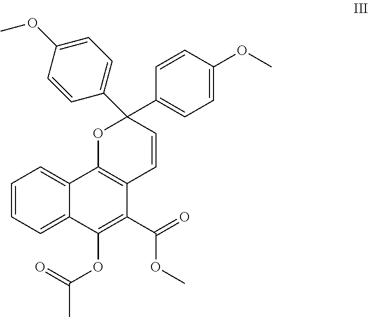

Furthermore, the particular novel naphthopyran compound of the present invention having formula II exhibits greater solubility in a polymer matrix and comparable photostability in relation to the conventional red-like photochromic compound having formula III.

Additional novel naphthopyran compounds of the present invention, which can be prepared in a similar fashion to the synthetic process described above, can include the novel naphthopyran compounds having formula IV through formula XXIII, as follows:

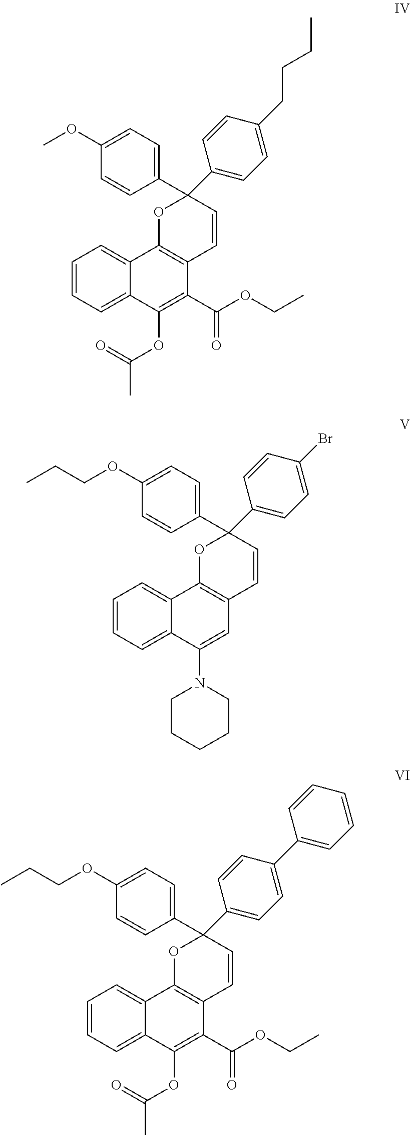

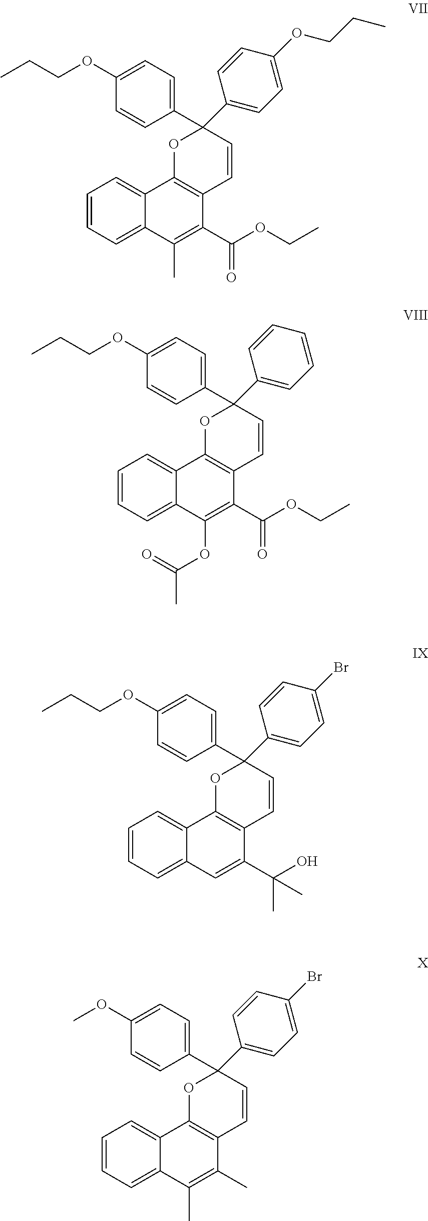

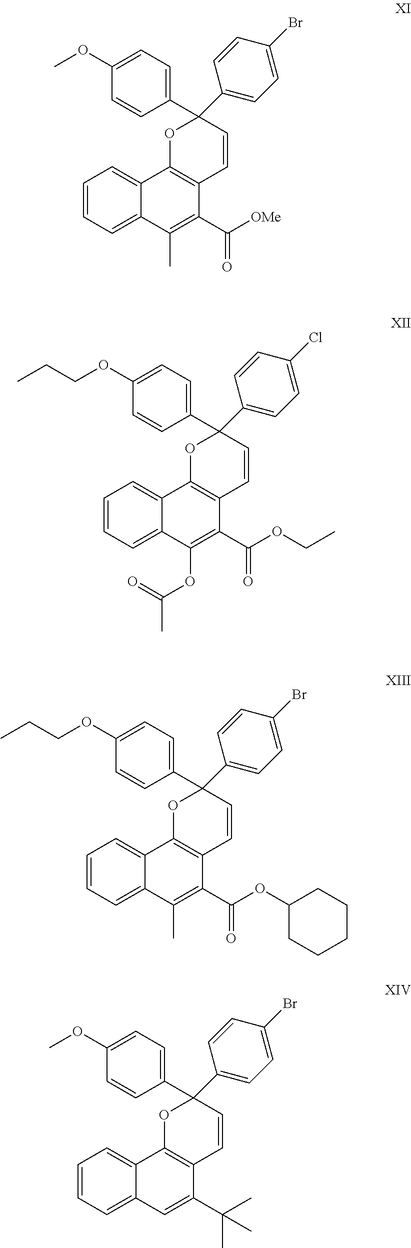

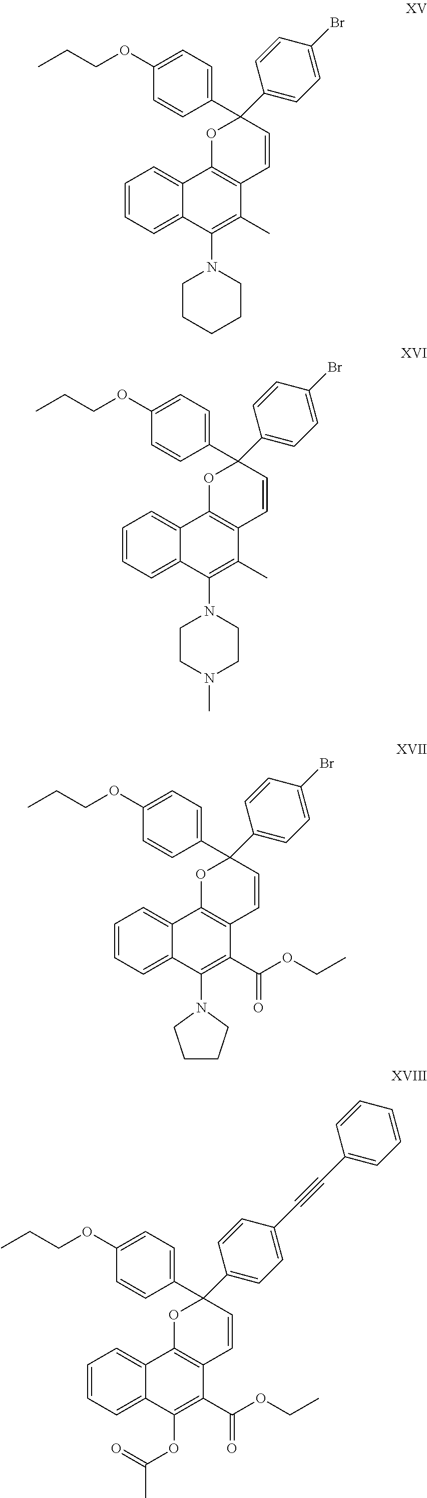

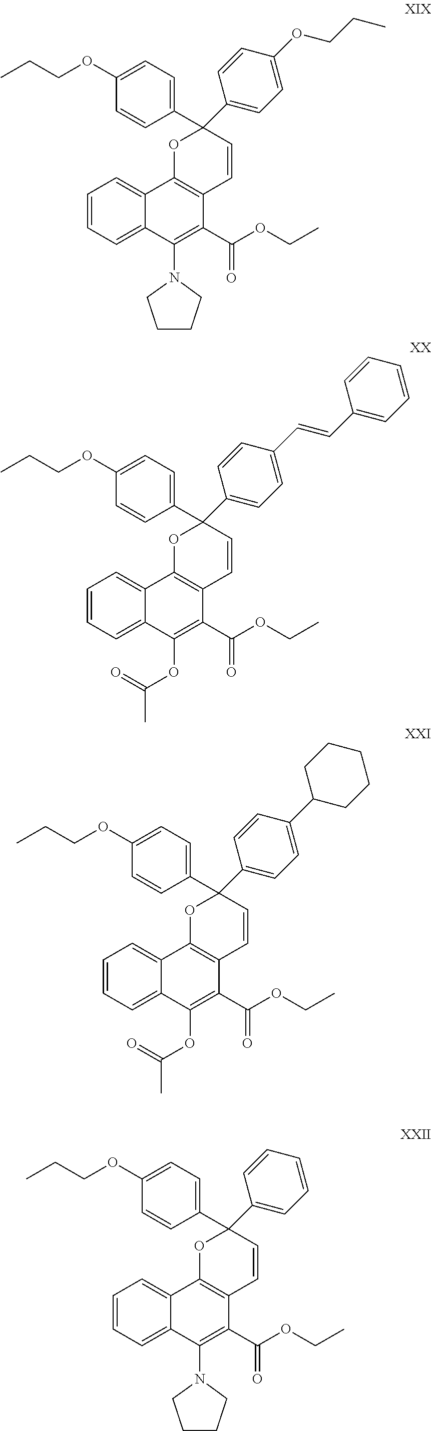

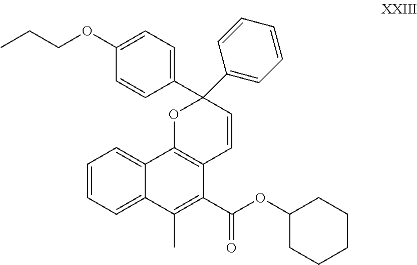 [SMILES: CCCOc1ccc(C2(c3ccccc3)C=Cc3c(C(=O)OC4CCCCC4)c(C)c4ccccc4c3O2)cc1]

As to particular embodiments, for example those embodied by formula II and formula IV through formula XVIII, the novel naphthopyran compounds of the present invention can have the following formula I:

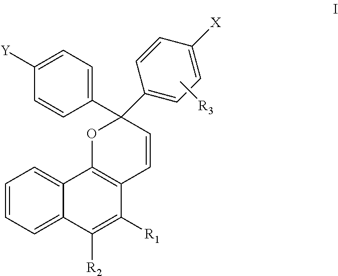

wherein:
R₁ is independently selected from alkyl, carbonyl, hydrogen, and hydroxy;
R₂ is independently selected from alkyl, amine, carbonyl, and hydrogen;
R₃ is independently selected from alkyl and hydrogen;
X is independently selected from alkenyl, alkoxy, alkyl, alkynyl, cyclohexayl, halo, hydrogen, and phenyl; and
Y is alkoxy.

As to particular embodiments, whether in R₁, R₂, or R₃, the carbonyl group can be a carboxyl group whereby the carboxyl group carbon, which is singly bonded to a first oxygen and doubly bonded to a second oxygen, is singly bonded directly to the corresponding ring.

Illustrative examples whereby R₁ can be a carboxyl group having the carboxyl group carbon singly bonded directly to the corresponding ring include the novel naphthopyran compounds having formulas II, IV, VI, VII, VIII, XI, XII, XIII, XVII, XVIII, XIX, XX, XXI, XXII, and XXIII.

As to particular embodiments, whether in R₁, R₂, or R₃, the carbonyl group can be a carboxyl group having the carboxyl group carbon singly bonded to a first oxygen and doubly bonded to a second oxygen, whereby the first oxygen is singly bonded directly to the corresponding ring.

Illustrative examples whereby R₂ can be a carboxyl group having the first oxygen singly bonded directly to the corresponding ring include the novel naphthopyran compounds having formulas II, IV, VI, VIII, XII, XVIII, XX, and XXI.

As to particular embodiments, whether in R₁, R₂, or R₃, the amine group can be an N-heterocyclic group. Illustrative examples whereby R₂ can be an N-heterocyclic group include the novel naphthopyran compounds having formulas V, XV, XVI, XVII, XIX, and XXII.

As to particular embodiments, R₁ and R₂ can be joined together to form a fused ring.

As to particular embodiments, X can be a halo group and, in particular, X can be bromine (Br), which can have an increased ability to leave in relation to chlorine (Cl) or fluorine (F). As an illustrative example, the increased ability to leave may be useful for converting the Br into another functional group, such as via a metal catalyzed reaction.

Illustrative examples whereby X can be a Br include the novel naphthopyran compounds having formulas II, V, IX, X, XI, XIII, XIV, XV, XVI, and XVII.

As to particular embodiments having X as a halo group, the halo group can be further transformed into an aryl group or an alkoxy group —OR₄, whereby R₄ can be a linear or branched C1-C12 alkyl.

In addition to novel naphthopyran compounds having photochromic properties whereby the novel naphthopyran compounds exhibit a red coloring when in a red-colored state achieved upon exposure to ultraviolet light, the present invention also relates to the incorporation of these novel naphthopyran compounds, whether singly or in combination depending upon the desired coloration, into host materials.

As non-limiting examples, host materials can include a plastics, plastic resins, plastic master batch concentrates, papers, textiles, inks, coatings, offset inks and coatings, metal decorating inks and coatings, UV radiation-curable inks and coatings, solvent-based inks and coatings, screen printing inks and coatings, gravure inks and coatings, lacquers, paints, adhesives, or the like.

As to particular embodiments, the host material having the novel naphthopyran compound can be used for a numerous and wide variety of purposes, which can include, as non-limiting examples, security printing, branding, interactive branding, brand protection, smart packaging, marketing, novelty printing, or the like, or combinations thereof.

As to particular embodiments, for incorporation into the host material, the novel naphthopyran compound can be microencapsulated. To form the microcapsules, the naphthopyran compound can be combined with a hydrophobic solvent to provide an internal phase about which a polymeric capsule wall can be built, whereby these methods are known to one of ordinary skill in the art of microencapsulation.

Further, as to particular embodiments, the microencapsulated naphthopyran compound can incorporated into a host material, such as one from the group listed above.

As can be easily understood from the foregoing, the basic concepts of the present invention may be embodied in a variety of ways. The invention involves numerous and varied embodiments of a photochromic compound and methods for making and using such photochromic compounds, including the best mode.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “combination” should be understood to encompass disclosure of the act of “combining”—whether explicitly discussed or not—and, conversely, were there effectively disclosure of the act of “combining”, such a disclosure should be understood to encompass disclosure of a “combination” and even a “means for combining”. Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the photochromic compounds herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A photochromic formulation comprising:
a naphthopyran compound which exhibits a red coloring when in a red-colored state achieved upon exposure to ultraviolet light, wherein said red coloring has a lambda max in a range of between about 490 nanometers to about 510 nanometers.

2. The photochromic formulation of claim 1, wherein said red coloring has a lambda max in a range of between about 490 nanometers to about 500 nanometers.

3. The photochromic formulation of claim 1, wherein said naphthopyran compound comprises the following formula: 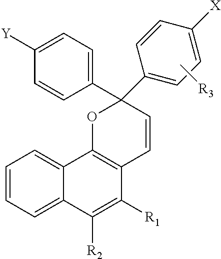
wherein:
R₁, R₂, and R₃ are each independently selected from acyloxy, alcohol, alkoxy, alkyl, amine, aryl, carbonyl, halo, heterocyclic, hydrogen, nitrile, and nitro;
X is independently selected from alkenyl, alkoxy, alkyl, alkynyl, amine, aryl, halo, and hydrogen; and
Y is independently selected from alkoxy, alkyl, amine, and hydrogen.

4. The photochromic formulation of claim 1, wherein said naphthopyran compound comprises the following formula: 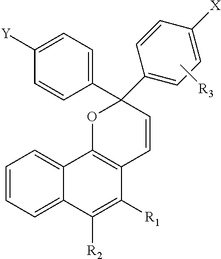
wherein:
R₁ is independently selected from alcohol, alkyl, carbonyl, and hydrogen;
R₂ is independently selected from alkyl, amine, carbonyl, and hydrogen;
R₃ is independently selected from alkyl and hydrogen;
X is independently selected from alkenyl, alkoxy, alkyl, alkynyl, cyclohexayl, halo, hydrogen, and phenyl; and
Y is alkoxy.

5. The photochromic formulation of claim 1, wherein said naphthopyran compound is selected from the group consisting of formulas II, IV, V, VI, VII, VIII, IX, X, XI, XII, XIII, XIV, XV, XVI, XVII, XVIII, XIX, XX, XXI, XXII, and XXIII.

6. The photochromic formulation of claim 3, wherein said carbonyl group comprises a carboxyl group having a carboxyl group carbon which is:
singly bonded to a first oxygen;
doubly bonded to a second oxygen; and
singly bonded directly to a corresponding ring.

7. The photochromic formulation of claim 1, wherein said naphthopyran compound is selected from the group consisting of formulas II, IV, VI, VII, VIII, XI, XII, XIII, XVII, XVIII, XIX, XX, XXI, XXII, and XXIII.

8. The photochromic formulation of claim 3, wherein said carbonyl group comprises a carboxyl group having a carboxyl group carbon which is:
singly bonded to a first oxygen; and
doubly bonded to a second oxygen;
wherein said first oxygen is singly bonded directly to a corresponding ring.

9. The photochromic formulation of claim 1, wherein said naphthopyran compound is selected from the group consisting of formulas II, IV, VI, VIII, XII, XVIII, XX, and XXI.

10. The photochromic formulation of claim 3, wherein said amino group of said R₁, said R₂, or said R₃ comprises an N-heterocyclic group.

11. The photochromic formulation of claim 1, wherein said naphthopyran compound is selected from the group consisting of formulas V, XV, XVI, XVII, XIX, and XXII.

12. The photochromic formulation of claim 3, wherein R₁ and R₂ are joined together to form a fused ring.

13. The photochromic formulation of claim 3, wherein X is Br.

14. The photochromic formulation of claim 1, wherein said naphthopyran compound is selected from the group consisting of formulas II, V, IX, X, XI, XIII, XIV, XV, XVI, and XVII.

15. The photochromic formulation of claim 3, wherein:
X is a halo group;
said halo group is further transformed into an aryl group or an alkoxy group —OR₄; and
R₄ is a linear or branched C1-C12 alkyl.

16. The photochromic formulation of claim 1, wherein said naphthopyran compound reversibly transitions from said red-colored state to a non-red colored state upon discontinuation of exposure to said ultraviolet light.

17. The photochromic formulation of claim 1, wherein said naphthopyran compound reversibly transition from said red-colored state to a non-red colored state upon an increase in temperature.

18. The photochromic formulation of claim 1, wherein said photochromic formulation is incorporated into a photochromic article.

19. The photochromic formulation of claim 1, wherein said photochromic formulation is incorporated into a photochromic coating.

20. The photochromic formulation of claim 1, wherein said photochromic formulation is incorporated into a photochromic ink.

21-26. (canceled)
